(19) United States
(12) Patent Application Publication
(10) Pub. No.: US 2009/0131785 A1
(43) Pub. Date: May 21, 2009

(54) VARIABLE OCCLUSIONAL BALLOON CATHETER ASSEMBLY

(75) Inventors: W. Anthony Lee, Gainesville, FL (US); Tomas D. Martin, Gainesville, FL (US); Nikolaus Gravenstein, Gainesville, FL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: University of Florida Research Foundation, Inc.

(21) Appl. No.: 12/271,380

(22) Filed: Nov. 14, 2008

Related U.S. Application Data
(60) Provisional application No. 60/988,427, filed on Nov. 15, 2007.

(51) Int. Cl. A61B 6/00; A61M 25/10

(52) U.S. Cl. 600/435; 604/96.01; 604/103.1; 604/103.07

(57) ABSTRACT

Embodiments of the present invention relate to inflatable balloon catheters and methods for deploying an inflatable balloon catheter. For example, one embodiment is directed to a catheter assembly that includes a catheter comprising proximal and distal ends and at least one longitudinal conduit extending between the proximal and distal ends. The catheter assembly also includes an inflatable balloon comprising proximal and distal ends and configured to be at least partially positioned within the longitudinal conduit when the balloon is uninflated. The inflatable balloon is further configured to be inflated when deployed from the conduit such that a diameter of the proximal end of the inflatable balloon remains substantially constant and a diameter of the distal end of the inflatable balloon is variable and capable of at least partially occluding a lumen.

### CROSS-REFERENCE TO RELATED APPLICATIONS

The present application claims priority from U.S. Provisional Application No. 60/988,427 filed Nov. 16, 2007, the contents of which are incorporated herein by reference.

### BACKGROUND

Intermittent periods of significant induced hypotension (with rapid return to normotension) may be necessary during placement of aortic and/or arterial endovascular devices. Induced hypotension may facilitate placement of endovascular devices within a vascular lumen where blood flow within the lumen may create difficulty in accurately positioning and implanting the device within a lumen. Since the majority of venous return to the heart comes via the inferior vena cava (IVC), precise and rapid induction of specific levels of hypotension can be accomplished by decreasing venous return to the heart. For example, operator-controlled hypotension can be achieved by: partially or completely occluding IVC blood flow, use of pharmacologic vasodilators, high airway pressures, deepened anesthetic, or a combination of such techniques. Current techniques for induced hypotension within the IVC are typically difficult to precisely control and technically inefficient.

Therefore, there is a need for systems and methods for variably and controllably occluding a lumen, such as the IVC, in order to at least partially occlude the IVC during a medical procedure.

### BRIEF SUMMARY

Embodiments of the present invention relate to inflatable balloon catheters and methods for deploying an inflatable balloon catheter. For example, one embodiment is directed to a catheter assembly that includes a catheter comprising proximal and distal ends and at least one longitudinal conduit extending between the proximal and distal ends. The catheter assembly also includes an inflatable balloon comprising proximal and distal ends and configured to be at least partially positioned within the longitudinal conduit when the balloon is uninflated. The inflatable balloon is further configured to be inflated when deployed from the conduit such that a diameter of the proximal end of the inflatable balloon remains substantially constant and a diameter of the distal end of the inflatable balloon is variable and capable of at least partially occluding a lumen. For instance, the inflatable balloon may be capable of at least partially occluding a lumen of a patient's right atrium (RA)/inferior vena cava (IVC) junction or the IVC.

According to one alternative aspect of the catheter assembly, the balloon catheter includes a plurality of longitudinal conduits, wherein one of the longitudinal conduits is configured to receive a guidewire therethrough. In addition, the guidewire may be configured to extend distally of the distal end of the inflatable balloon. The catheter assembly may further include a radiopaque marker positioned at the proximal and/or distal ends of the inflatable balloon. The balloon catheter may include a plurality of graduated markings. Moreover, the inflatable balloon may be substantially conical in shape. For example, the diameter of the proximal end of the inflatable balloon may be smaller than the diameter of the distal end of the inflatable balloon, such as about 1.5 to 3.5 times smaller. The catheter assembly may additionally include a mechanism for maintaining the inflatable balloon in a substantially fixed position relative to the introducer conduit and/or a fastening mechanism configured to engage the proximal end of the inflatable balloon catheter so as to prevent distal movement of the inflatable balloon within the lumen. The inflatable balloon may be configured to be inflated with a mixture of contrast media and saline solution.

Another embodiment of the present invention relates to a method for deploying an inflatable balloon catheter such that a diameter of the proximal end of the inflatable balloon remains substantially constant and a diameter of the distal end of the inflatable balloon is variable and capable of at least partially or even completely occluding a lumen. Furthermore, one embodiment of a balloon for use within a catheter assembly includes an inflatable body portion that is adapted so that, when said body portion is inflated, said body portion is substantially in the shape of a conical section (e.g., a cone or truncated cone shape).

An additional embodiment is directed to a method for at least partially occluding a patient's RA/IVC junction. The method includes: (A) positioning a balloon at least partially within said patient's RA, wherein the balloon being adapted so that, when the balloon is inflated, the balloon is substantially in the shape of a conical section (e.g., a cone shape); (B) while said balloon is positioned at least partially within the patient's RA (e.g., substantially entirely or entirely within the RA), inflating the balloon until the balloon is in an inflated state in which the balloon is substantially in the shape of a conical section (e.g., a cone shape); (C) while the balloon is in the inflated state, positioning the balloon so that: (1) an apex of the balloon is positioned adjacent the patient's RA/IVC junction; and (2) a base portion of the balloon is positioned distally away from both the apex of the balloon and the RA/IVC junction; and (D) after the Step (C), positioning the balloon so that: (1) the apex of said balloon is disposed within the patient's IVC; (2) the base portion of the balloon is disposed within the RA; and (3) at least a side wall of the balloon at least partially occludes the patient's RA/IVC junction.

### BRIEF DESCRIPTION OF THE DRAWINGS

Having thus described the invention in general terms, reference will now be made to the accompanying drawings, which are not necessarily drawn to scale, and wherein:

FIG. 1 shows a cross-sectional view of a catheter positioned adjacent a patient's IVC according to one embodiment of the invention.

FIG. 2 depicts a cross-sectional view of a catheter for occluding a lumen according to an embodiment of the invention.

FIG. 3 is a graphical representation of the relationship between volume and pressure within an inflatable balloon according to a particular embodiment of the invention.

FIG. 4 is a graphical representation of the relationship between diameter and volume of an inflatable balloon according to an embodiment of the invention.

### DETAILED DESCRIPTION OF VARIOUS EMBODIMENTS OF THE INVENTION

The present invention will now be described more fully hereinafter with reference to the accompanying drawings, in which various embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

### Overview

Various embodiments of the present invention are configured to provide techniques for at least partially occluding a lumen. For example, one embodiment of the present invention provides a catheter assembly capable of at least partially occluding a patient's IVC, such as to induce hypotension during a medical procedure. The catheter assembly generally includes a balloon catheter having a conduit configured to allow inflation and deflation of an inflatable balloon. The inflatable balloon is configured to be deployed from the conduit and adjacent the lumen so that a narrow, proximal end of the balloon (which, in various embodiments, is generally conically shaped) is immediately adjacent the lumen and so that the wider, distal end of the balloon is spaced distally apart from the balloon's proximal end. An operator then retracts the balloon toward the proximal end of the balloon catheter until: (1) the narrow end of the balloon moves through the lumen; and (2) a side portion of the balloon that is between the balloon's proximal and distal ends engages the perimeter of the lumen to thereby at least partially occlude the lumen.

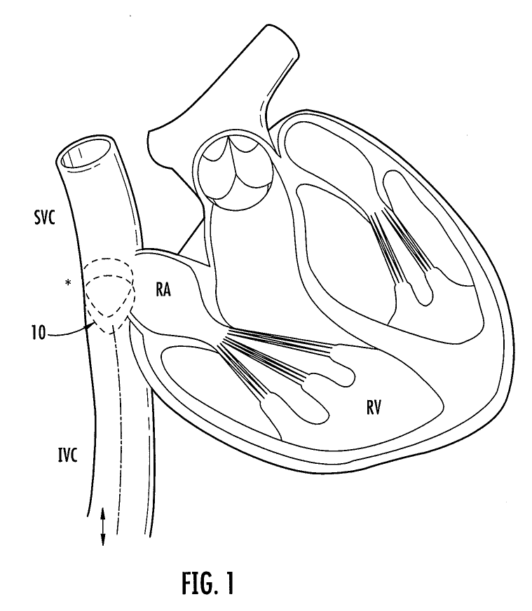

For example, in a particular embodiment of the invention, a substantially conically shaped balloon 12 is first inflated in a patient's right atrium (as depicted by the upper dashed illustration of balloon 12 in FIG. 1), and then retracted so that the narrow, proximal end of the balloon 12 is disposed within the patient's IVC, and the distal end of the balloon 12 (which is preferably wider in diameter than the balloon's proximal end) is disposed within the patient's right atrium (as depicted by the lower dashed illustration of balloon 12 in FIG. 1). As shown in FIG. 1, in this position, the balloon 12 serves to at least partially occlude the lumen at the right atrium/IVC junction.

In particular embodiments, the operator adjusts how far to retract the balloon 12 into the IVC by monitoring the patient's blood pressure and may also choose to monitor the pressure inside the patient's right atrium (central venous pressure). When the desired blood pressure is reached, the operator stops retracting the balloon 12 into the IVC, and uses a fastening mechanism (e.g., a clip) to maintain the balloon 12 in the desired position.

According to one embodiment, hypotension can be induced safely and precisely by partially or completely occluding IVC blood flow by varying the diameter of the inflatable balloon to which the balloon is inflated.

### More Detailed Description

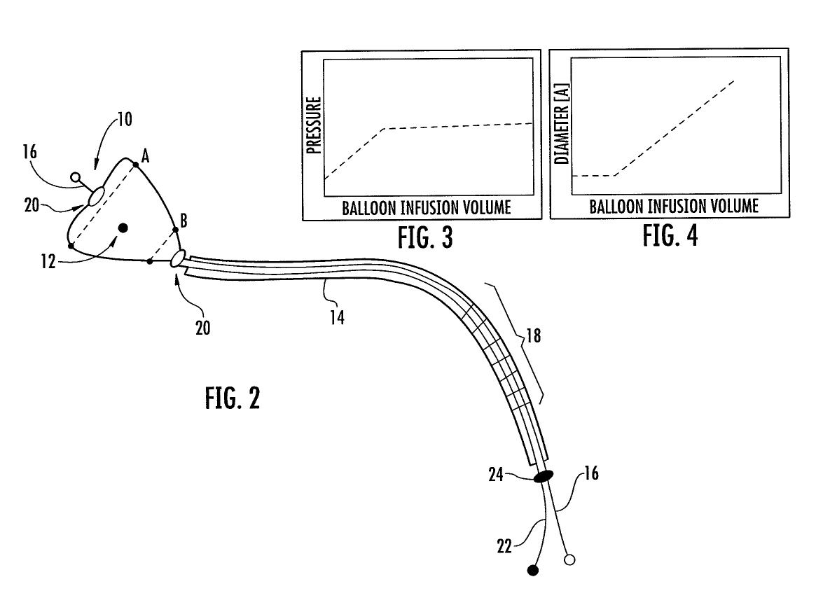

As noted above, various embodiments of the present invention provide techniques for occluding a lumen. FIGS. 1 and 2 depict an exemplary embodiment of a catheter assembly 10 for at least partially occluding a lumen with an inflatable balloon 12. The catheter assembly 10 generally includes a balloon catheter 14 for receiving the inflatable balloon 12 and is capable of receiving other instruments therein, such as a guidewire 16. The catheter assembly 10 is capable of occluding a variety of vascular and non-vascular lumens, such as the IVC. Thus, the diameter of the inflatable balloon 12 may be adjusted by balloon size or inflation volume to occlude lumens of various sizes and configurations.

Balloon Catheter

According to one embodiment of the present invention, the balloon catheter 14 is a flexible tubular member having proximal and distal ends. The balloon catheter 14 is typically somewhat flexible so as to allow the balloon catheter to be introduced transvenously and to adapt to a variety of contours. The balloon catheter may comprise any of a variety of flexible materials, such as a polymeric material (e.g., polytetrafluoroethylene, polyurethane, or silicone).

In particular embodiments, the balloon catheter 14 includes one or more longitudinal conduits extending between its proximal and distal ends that are capable of inflating and deflating the inflatable balloon 12 and/or receiving other instruments. The inflatable balloon 12, guidewire 16, or other instruments may extend through a central longitudinal conduit within the balloon catheter 14 or through respective longitudinal conduits. Alternatively, the balloon catheter 14 may include a Luer connector for facilitating the insertion of various instruments or administration of fluids through the balloon catheter 14. The inflatable balloon 12 and other instruments positioned within respective longitudinal conduits may be axially displaceable so as to be capable of being inserted through the proximal end of a longitudinal conduit and distally of the distal end of the longitudinal conduit, such as shown by the guidewire 16 extending distally relative to the balloon 12 shown in FIG. 2. In addition, the proximal ends of the inflatable balloon 12, guidewire 16, or other instruments may extend proximally out of the proximal end of the balloon catheter 14 so that an operator may manipulate the balloon, guidewire, or other instrument. It is understood that the catheter assembly 10 may also include an actuator or other mechanism to facilitate deployment of the balloon 12 and/or operation of the instruments.

The balloon catheter 14 may include a plurality of graduated markings 18 (which may be, for example, printed on the balloon catheter so that they are visible to the naked eye or radiographically visible) to facilitate visual monitoring of the axial displacement of the balloon catheter. For example, FIG. 2 shows that graduated markings 18 may be positioned near one or more of the ends of the balloon catheter 14. However, it is understood that the balloon catheter 14 may include markings 18 at any desired location to facilitate monitoring of the displacement thereof.

Furthermore, the balloon catheter 14 may include a valve for preventing leakage of blood or other fluid proximally out of the balloon catheter. For example, the valve may be a hemostatic valve, or any valve known to those of skill in the art, that prevents fluid leakage while also allowing instruments to pass through the balloon catheter 14.

Guidewire

The catheter assembly 10 may include a longitudinal conduit for receiving a guidewire 16 therethrough. The guidewire 16 may be employed to facilitate placement of the catheter assembly 10 within the lumen by first inserting the guidewire into the target vessel and then sliding the balloon catheter 14 over the guidewire to a desired location adjacent the lumen. The guidewire 16 may be left within the balloon catheter 14 while the balloon 12 is inflated, or the guidewire may be removed once the balloon catheter 14 is positioned within a desired location.

The balloon catheter 14 may also include one or more additional longitudinal conduits for receiving various instruments. For example, the balloon catheter 14 may include a longitudinal conduit configured for receiving one or more instruments - such as a pressure transducer for measuring atrial pressure, or to receive a syringe or other device for injecting a fluid, such as a contrast media, or fluid within the lumen. For instance, a contrast media may be injected out of the distal end of the balloon catheter 14 for facilitating right atrial angiography. The pressure transducer may be employed to monitor pressure in or on the right atrium and/or IVC prior to and resulting from inflating the balloon 12 so as to reduce the risk or rupturing the atrium or IVC. In particular embodiments, the pressure transducer conduit may be positioned so that it is spaced distally apart from the distal end of the balloon 12 as the pressure transducer monitors the pressure within the patient's right atrium.

Inflatable Balloon

In various embodiments, the balloon 12 comprises a compliant material that is configured to expand upon an increase in volume within the balloon 12. For example, a contrast media, a contrast and saline mixture, air, or any suitable liquid or gas may be injected within the balloon 12 to inflate or otherwise increase the volume of the balloon. In addition, injecting a radiopaque material, such as a contrast media, may facilitate monitoring of the size and position of the balloon 12 within the lumen. The balloon 12 may include radiopaque markers 20 on its proximal and/or distal end to monitor the size and position of the balloon 12.

In particular embodiments, in an uninflated state, the balloon 12 is configured to be positioned against the balloon catheter 14. In particular embodiments, a conduit 22 or other inflating mechanism is coupled to the balloon 12 and is configured to: (1) inject a gas or fluid into the balloon for inflating the balloon 12; and/or (2) withdraw a gas or fluid from the balloon 12 to deflate the balloon 12. The proximal end of the conduit 22 may include a valve stopcock, or similar device, to prevent the balloon 12 from deflating, as well as to provide an operator with the ability to control the amount that the balloon is deflated.

In particular embodiments, when inflated, the balloon 12 is generally conical in shape, as shown in FIG. 2. The distal end of the balloon 12 expands outwardly from the balloon's proximal end. The expanded balloon 12 may be various sizes for accommodating different lumens, and according to one embodiment, the expanded diameter indicated by “B” in FIG. 2 is about 10 mm, while the expanded diameter indicated by “A” may be varied between about 15-35 mm. Thus, the expanded diameter at or near the distal end of the balloon 12 may be varied while the diameter at or near the proximal end of the balloon may remain substantially constant. It should be understood that, in various embodiments, the balloon 12 may be any of various shapes and sizes for conforming to various body lumens. Applicants note that a substantially conically shaped balloon may provide a “wedge” for facilitating occlusion of the lumen, as explained in further detail below. Applicants further note that balloons of other shapes may also provide such a “wedge”. Such shapes include, for example, a substantially truncated cone shape (e.g., a cone with a truncated narrow end).

The balloon 12 may comprise a material that exhibits predictable compliance characteristics. For example, the balloon 12 may be a flexible material such as silicone or latex. Namely, FIG. 3 depicts the relationship of volume and pressure within the balloon 12, wherein an increase in volume within the balloon 12 initially increases pressure within the balloon 12, but the pressure within the balloon 12 remains substantially constant with a further increase in volume until the compliant volume is reached. FIG. 4 shows that, in various embodiments, an initial increase in volume does not result in a change in diameter “A” of the balloon 12, but after a predetermined threshold in volume is reached, a further increase in volume results in an increase in diameter “A.” Therefore, the conical design and low-pressure employed may reduce the risk of right atrial rupture or IVC disruption.

Fastening Mechanism

The catheter assembly 10 may also include a fastening mechanism 24 for securing the balloon 12, guidewire 16, and/or other instrument(s) in position. In particular, after inflating the balloon 12 to a desired diameter (and/or positioning the balloon 12 in the desired position), the physician may use the fastening mechanism 24 to lock the balloon 12 in position (e.g., relative to the introducer conduit through which the balloon catheter 14 is placed) to prevent the balloon 12 from moving further toward the heart due to blood flow. Thus, the balloon 12 may be inflated to a diameter that satisfactorily occludes the lumen or may be retracted so that it satisfactorily occludes the lumen and then be secured so that the operator is free to use his or her hands to perform additional procedures.

The fastening mechanism 24 may be any device configured to secure the balloon 12 and/or the balloon catheter in a desired position. For example, the fastening mechanism 24 may be a compression screw (or any other suitable fastening device) that extends radially within a standard catheter introducer associated with the balloon catheter 14 and that is configured to engage the conduit 22 to prevent movement of the balloon catheter 14 (and therefore the balloon 12) toward the patient's heart (which would typically result in the balloon 12 being less occlusive). A further example includes a clip on the balloon catheter 14 that secures the balloon catheter 14 to the conduit 22, which thereby prevents movement of the balloon catheter 14 toward the patient's heart.

In various embodiments, the fastening mechanism 24 may be a Tuohy-Borst mechanism. For instance, in particular embodiments, a Tuohy-Borst mechanism may be threadably engaged with the proximal end of the introducer 14 and configured to secure the balloon 12 and/or other instruments in position.

### Exemplary Method

In particular embodiments, the catheter assembly 10 may be used for at least partially occluding a body lumen. According to one embodiment, the catheter assembly 10 may be used for occluding the IVC, such as to induce hypotension during a medical procedure. For example, in various embodiments, the catheter assembly 10 may be used to occlude the IVC during a stenting procedure in the aorta, or any artery. Thus, the inflatable balloon 12 may first be positioned in the right atrium and then manually retracted at least partially into the IVC to achieve a graded obstruction to right atrial inflow, as shown in FIG. 1.

In use, according to one embodiment, the catheter assembly 10 is delivered using a transfemoral venous approach. In particular, the femoral vein is accessed via the patient's groin, and a guidewire 16 is positioned through the femoral vein and into the patient's IVC. The balloon catheter 14 may then be advanced independently or over the guidewire and into the patient's IVC. The position of the balloon catheter 14 may be monitored using various imaging techniques, such as fluoroscopy or angiography. For example, contrast media may be injected through one of the longitudinal conduits defined in the balloon catheter 14 and out of the distal end thereof. Typically, the balloon catheter 14 would be located such that its distal end is in the right atrium, as shown in FIG. 1. The balloon catheter 14 is then retracted in order to deploy the balloon 12 within the lower right atrium and proximal IVC.

The position of the balloon 12 may be indicated by proximal and/or distal radiopaque markers 20, and/or by inflating the balloon with contrast media. Longitudinal displacement of the balloon catheter 14 may also be monitored by the graduated markings 18.

Next, the balloon 12 is inflated to a desired volume within the right atrium. The operator may utilize a pressure transducer positioned within the balloon end of the balloon catheter 14 to monitor the pressure on the right atrium. When the balloon 12 is inflated to a desired distal diameter “A,” the surgeon may then retract the balloon 12 so as to at least partially retract the balloon into the IVC (the proximal retraction of the balloon 12 from the SVC or right atrium to the IVC is indicated by dashed lines in FIG. 1) until the patient's blood pressure reaches a desired level. In various embodiments, when the balloon 12 is in this position, the balloon 12 is “wedged” in the junction between the IVC and the RA, so that the narrow proximal end of the balloon 12 is disposed within the IVC, and so that the wider distal end of the balloon 12 is disposed within the RA.

Next, the operator may then use a fastening mechanism 24 in order to secure the balloon catheter 14 in position. While the IVC is partially or completely occluded, which preferably results in induced hypotension, the operator may then perform a medical procedure in the aortic or arterial circulation. For example, the operator may implant a stent in the aorta or an iliac artery. After performing the procedure, the balloon 12 may be deflated and retracted, and the balloon catheter 14 may be removed from the patient's body.

It should be understood that the aforementioned discussion regarding the procedure for occluding a lumen is not meant to be limiting, as the specific procedure employed may depend on the operator or the specific medical procedure to be performed. For example, the specific order of steps used to occlude the lumen may vary, and/or an operator may use various visualization techniques to aid in positioning the catheter assembly 10 in the lumen.

### CONCLUSION

Many modifications and other embodiments of the invention will come to mind to one skilled in the art to which this invention pertains having the benefit of the teachings presented in the foregoing descriptions and the associated drawings. For example, as will be understood by one skilled in the relevant field in light of this disclosure, the invention may take form in a variety of different mechanical and operational configurations. Therefore, it is to be understood that the invention is not to be limited to the specific embodiments disclosed and that modifications and other embodiments are intended to be included within the scope of the appended exemplary concepts. Although specific terms are employed herein, they are used in a generic and descriptive sense only and not for the purposes of limitation.

That which is claimed:

1. A catheter assembly comprising:
a catheter comprising proximal and distal ends and at least one longitudinal conduit extending between the proximal and distal ends; and
an inflatable balloon comprising proximal and distal ends and configured to be at least partially positioned within the longitudinal conduit when the balloon is uninflated, wherein:
the inflatable balloon is further configured to be inflated when deployed from the conduit such that a diameter of the proximal end of the inflatable balloon remains substantially constant and a diameter of the distal end of the inflatable balloon is variable and capable of at least partially occluding a lumen of a patient's right atrium (RA)/inferior vena cava (IVC) junction or the IVC.

2. The catheter assembly of claim 1, wherein the balloon catheter comprises a plurality of longitudinal conduits, and wherein one of the longitudinal conduits is configured to receive a guidewire therethrough.

3. The catheter assembly of claim 2, wherein the guidewire is configured to extend distally of the distal end of the inflatable balloon.

4. The catheter assembly of claim 1, further comprising a radiopaque marker positioned at the proximal and/or distal ends of the inflatable balloon.

5. The catheter assembly of claim 1, wherein the balloon catheter comprises a plurality of graduated markings.

6. The catheter assembly of claim 1, wherein the inflatable balloon is substantially conical in shape.

7. The catheter assembly of claim 6, wherein the diameter of the proximal end of the inflatable balloon is smaller than the diameter of the distal end of the inflatable balloon.

8. The catheter assembly of claim 7, wherein the diameter of the distal end of the inflatable balloon is variable to be between about 1.5 to 3.5 times larger than the diameter of the proximal end of the inflatable balloon.

9. The catheter assembly of claim 1, further comprising a mechanism for maintaining the inflatable balloon in a substantially fixed position relative to the introducer conduit.

10. The catheter assembly of claim 1, wherein the inflatable balloon is configured to be inflated with a mixture of contrast media and saline solution.

11. The catheter assembly of claim 1, further comprising a fastening mechanism configured to engage the proximal end of the inflatable balloon catheter so as to prevent distal movement of the inflatable balloon within the lumen.

12. A method for deploying an inflatable balloon catheter such that a diameter of the proximal end of the inflatable balloon remains substantially constant and a diameter of the distal end of the inflatable balloon is variable and capable of at least partially or even completely occluding a lumen.

13. A balloon for use within a catheter assembly, said balloon comprising an inflatable body portion that is adapted so that, when said body portion is inflated, said body portion is substantially in the shape of a conical section.

14. The balloon of claim 13, wherein said conical section is a cone.

15. The balloon of claim 13, wherein said conical section is a truncated cone.

16. A method of at least partially occluding a patient's right atrium (RA)/inferior vena cava (IVC) junction, said method comprising the steps of:
(A) positioning a balloon at least partially within said patient's RA, said balloon being adapted so that, when said balloon is inflated, said balloon is substantially in the shape of a conical section;
(B) while said balloon is positioned at least partially within said patient's RA, inflating said balloon until said balloon is in an inflated state in which said balloon is substantially in the shape of a conical section;
(C) while said balloon is in said inflated state, positioning said balloon so that: (1) an apex of said balloon is positioned adjacent said patient's RA/IVC junction; and (2) a base portion of said balloon is positioned distally away from both said apex of said balloon and said RA/IVC junction; and
(D) after said Step (C), positioning said balloon so that: (1) said apex of said balloon is disposed within said patient's IVC; (2) said base portion of said balloon is disposed within said RA; and (3) at least a side wall of said balloon at least partially occludes said patient's RA/IVC junction.

17. The method of claim 16, wherein:
said balloon is adapted so that, when said balloon is inflated, said balloon is substantially in the shape of a cone; and
when said balloon is in said inflated state, said balloon is substantially in the shape of a cone.

18. The method of claim 16, wherein said Step (B) is executed while said balloon is positioned substantially entirely within said patient's RA.

19. The method of claim 16, wherein said Step (B) is executed while said balloon is positioned entirely within said patient's RA.
